(19) United States
(12) Patent Application Publication
(10) Pub. No.: US 2012/0165527 A1
(43) Pub. Date: Jun. 28, 2012

(54) PROCESS FOR THE PREPARATION OF PURE PALIPERIDONE

(75) Inventors: Buchi Reddy Reguri, Chennai (IN); Muthulingam Arunagiri, Chennai (IN); Saravanakumar Kasiyappan Gurusamy, Chennai (IN); Kondalarao Ponnapally, Chennai (IN)

(73) Assignee: ORCHID CHEMICALS & PHARMACEUTICALS LTD., Chennai, Tamil Nadu (IN)

(21) Appl. No.: 13/388,173

(22) PCT Filed: Aug. 4, 2010
(86) PCT No.: PCT/IB2010/001924

§ 371 Date: Mar. 2, 2012

(30) Foreign Application Priority Data
Aug. 4, 2009 (IN) 1852/CHE/2009

(51) Int. Cl. C07D 471/04

(52) U.S. Cl. 544/282

(57) ABSTRACT

The present invention relates to an improved process for the preparation of pure Paliperidone of formula (I). The present invention more specifically provides an improved process for the preparation of pure Paliperidone which may contain impurities in the acceptable level of pharmacopoeia requirement specifically 3-{2-[4-(5-Fluoro-benzo[d]isoxazol-3-yl]-piperidin-1-yl]-ethyl}-2-methyl-7,8-dihydro-6H-pyrido[1,2-a]pyrimidine-4,9-dione of Formula (IV).

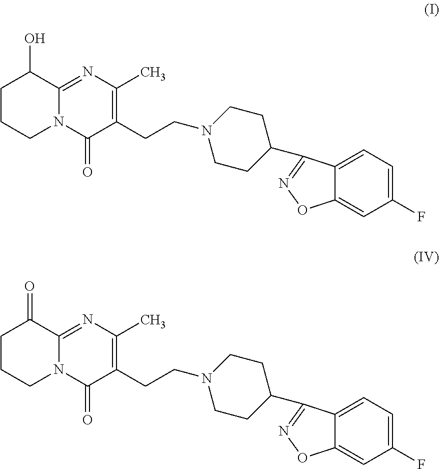

### FIELD OF THE INVENTION

The present invention relates to an improved process for the preparation of substantially pure Paliperidone.

### BACKGROUND OF THE INVENTION

Paliperidone, 3-[2-[4-(6-fluorobenzo[d]isoxazol-3-yl)-1-piperidyl]-7-hydroxy-4-methyl-1,5-diazabicyclo[4.4.0]deca-3,5-dien-2-one, is a 5-HT antagonist belonging to the chemical class of benzisoxazole derivatives and is present as a racemic mixture having the following structural formula:

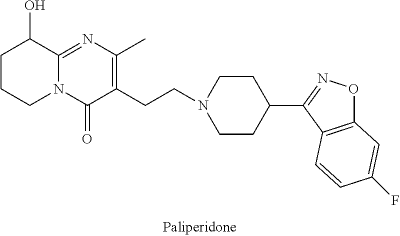

Paliperidone is a metabolite of Risperidone, marketed under the name, Invega®. Paliperidone is a psychotropic agent approved in the United States for the treatment of schizophrenia.

A process for the synthesis of Paliperidone is described in U.S. Pat. No. 5,158,952. As disclosed in this patent, the Paliperidone is prepared via the intermediate 342-chloroethyl)-6,7,8,9-tetrahydro-9-hydroxy-2-methyl-4H-pyrrido[1,2-a]pyrimidi-4-one (CMHTP). The process includes the condensation of 3-(2-chloroethyl)-6,7,8,9-tetrahydro-9-hydroxy-2-methyl-4H-pyrrido[1,2-a]pyrimidi-4-one (CMHTP) with 6-fluoro-3-piperidino-1,2-benisoxazol (FBIP) in presence of a secondary organic amine and an alcohol to get the crude Paliperidone. The obtained crude product is purified by column chromatography and then crystallized in acetone. The product obtained from first crystallization is under-go second re-crystallization using 2-propanol to get the pure Paliperidone with 21% yield.

Process for the synthesis of intermediates of Paliperidone is described also in U.S. Pat. No. 5,688,799.

The processes described in the above publications have many disadvantages like long reaction time, recovery stages and poor quality along with low chemical yields, making their application in the industry very hard. Moreover, the product obtained from process as disclosed in prior art have a very high content of impurities such as N-oxide of Paliperidone as well as carboxylate of Paliperidone. Hence, there is a need in the art for a new/improved process for preparing substantially pure Paliperidone, which is simple, cost effective, eco-friendly and commercially suitable process by over coming the problems encountered in the above prior art process.

### OBJECTIVE OF THE INVENTION

An object of the invention is to produce an economically and industrially viable process for producing paliperidone.

### SUMMARY OF THE INVENTION

The present invention relates to an improved process for the preparation of substantially pure Paliperidone, by condensing 3-(2-chloroethyl)-6,7,8,9-tetrahydro-9-hydroxy-2-methyl-4H-pyrrido[1,2-a]pyrimidi-4-one (CMHTP) with 6-fluoro-3-piperidino-1,2-benisoxazol (FBIP) or salt thereof in presence of a tertiary organic amine as a base in solvent and optionally in presence of phase transfer catalyst/N,N-dimethylamino pyridine (DMAP).

Another objective of the present invention is to minimize the level of impurities like Paliperidone N-oxide and Paliperidone carboxylate in desired final product by recrystallization in alcohol, ketone, and mixture of ketone or alcohol with water.

Thus the process of the present invention is simple, reproducible, cost-effective, eco-friendly non-hazardous and hence can be well suited for large-scale production.

### DESCRIPTION OF FIGURES

FIG. 1 is an X-ray powder diffractogram of a Paliperidone crystalline form prepared by according to the present invention.

FIG. 2 is a differential scanning calorimetric thermogram of a Paliperidone crystalline form prepared by according to the present invention.

FIG. 3 is an infrared absorption spectrum of a Paliperidone crystalline form prepared by according to the present invention.

### DETAILED DESCRIPTION OF THE INVENTION

Accordingly, the present invention provides for a process for the preparation of Paliperidone comprising the condensing the 3-(2-chloroethyl)-6,7,8,9-tetrahydro-9-hydroxy-2-methyl-4H-pyrrido[1,2-a]pyrimidi-4-one (CMHTP) (I) and 6-fluoro-3-piperidino-1,2-benisoxazol (FBIP) (II) or salt thereof in presence of tertiary organic amine as a base and solvent system to provide the crude Paliperidone (III) as depicted in scheme 1.

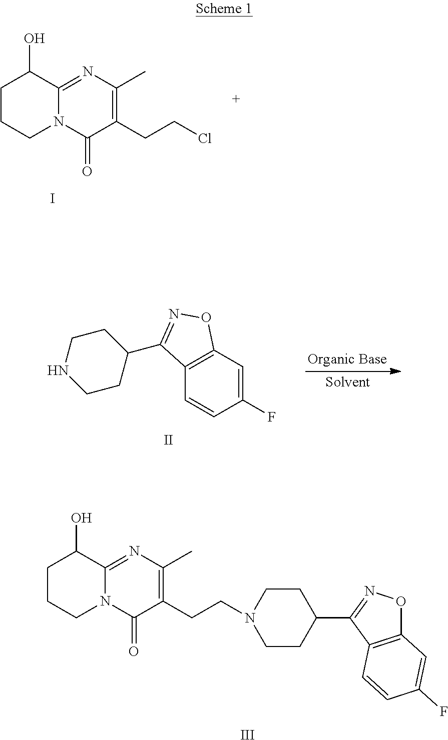

Preferably, the solvents used herein is selected from the group consisting of but not limited to C₁₋₆ straight or branched chain alcohol, ketone, ester, polar aprotic solvent and ether such as methanol, ethanol, n-propyl alcohol, isopropyl alcohol, n-butyl alcohol, t-butyl alcohol, acetone, ethyl methyl ketone, ethyl acetate, methyl acetate, dimethylformamide, dimethyl acetamide, N-methylpyrrolidinone, diethyl ether, diisopropyl ether and methyl ether. The tertiary organic amine base used herein is selected from the group consisting of but not limited to triethyl amine, diisopropyl ethyl amine, trimethyl amine, diisopropyl methyl amine, N-methyl morpholine and diethyl methyl amine, preferably diisopropyl ethyl amine. The above reaction can also be done in presence of phase transfer catalyst or dimethylamino pyridine (DMAP). The phase transfer catalyst used herein is selected from the group consisting of but not limited to trialkylphenylmethylammonium, tetraalkylammonium, tetraalkylphosphonium, tetraarylphosphonium halide, hydroxide, hydrogen sulfate and the like.

The crude Paliperidone is further purified by treating it with alcohol, ketone or their mixture with water to get the substantially pure Paliperidone free from the N-oxide as well as carboxylate impurity. The ketone used is acetone, methyl ethyl ketone, methyl isobutyl ketone, preferably acetone and the alcohol used is isopropyl alcohol, methanol, ethanol, n-propyl alcohol, preferably isopropyl alcohol. The product obtained from above process optionally further purified by treating it with alcohol or mixture of water and alcohol. The alcohol used herein is methanol, ethanol, n-propyl alcohol, butyl alcohol and isopropyl alcohol, preferably isopropyl alcohol.

### ADVANTAGES

The present invention avoids the column chromatography for the isolation of paliperidone as disclosed in prior art as well as the product obtained from above process is free from N-oxide and carboxylate impurities which were present in prior art processes.

The present invention is illustrated by the following examples, which are not intended to limit the effective scope of the invention.

### EXAMPLES

### Example 1

### Preparation of Crude Paliperidone

A 200 ml flask equipped with a mechanical stirrer, reflux condenser was charged under nitrogen with FBIP.HCl (10 gm), CMHTP (12.6 gm), Diisopropyl ethyl amine (11.08 gm), and 50 ml methanol. The suspension was heated to reflux 67-68° C. for 10 to 12 hours under Nitrogen atmosphere. After completion of reaction the reaction was cooled to room temp for 30 min, then filtered under reduced pressure and rinsed with methanol (3×10 ml). The resultant solid was slurried with 40 ml water, filtered to obtain Paliperidone crude wet cake.

### Crude Paliperidone: 99.4%

### N-Oxide Impurity: 0.08%

Carboxylate impurity: Not detected

### Example 2

### Preparation of Pure Paliperidone

A 500 ml flask equipped with a mechanical stirrer was charged with crude product (14.54 gm) of example 1 and dichloromethane (230 ml). The reaction mass was stirred at 25° C. to 30° C. until the content dissolves followed by cooling to room temperature. The pH 3.0+0.2 was adjusted of the reaction mixture using the 10% w/v dilute HCl and stirred. Layers were separated and the acetone (159 ml) was added to aqueous layer with stirring. The reaction mass was then basified to pH 8.0+0.2 by using 10% w/v aqueous NaHCO₃ solution. The obtained precipitates were filtered and suck dried. The wet product is then dried in vacuum at 50-55° C.

### Crude Paliperidone: 99.6%

### N-Oxide Impurity: 0.05%

Carboxylate impurity: Not detected

### Example 3

### Preparation of Highly Pure Paliperidone

A 500 flask equipped with mechanical stirrer and reflux condenser was charged with IPA (210 ml) and water (4.2 ml). The reaction mass was heated to 40° C. to 45° C. followed by the addition of product obtained from example 2 and stirred. Filter the reaction mass after 2 hour and material obtained was suck dried. The wet cake was then dried under vacuum at 50-55° C.

### Crude Paliperidone: 99.8%

### N-Oxide Impurity: 0.03%

Carboxylate impurity: Not detected

### Example 4

### Preparation of Crude Paliperidone Using Phase Transfer Catalyst

A 100 ml Flask equipped with a mechanical stirrer, reflux condenser was charged under nitrogen with FBIP.HCl (5 gm), CMHTP (6.29 gm), Diisopropyl ethyl amine (5.54 grn), tetra n-butyl ammonium bromide (0.03 gm) and 25 ml methanol. The suspension was heated to reflux 67-68° C. for 10 to 12 hours. After completion of reaction the reaction was cooled to room temperature followed by filtration under reduced pressure and rinsed with methanol (3×5 ml). The resultant solid was slurried with 200 ml water, filtered and dried at 60° C. under reduced pressure for 6 to 8 hour to obtain Paliperidone.

### Crude Paliperidone: 99.6%

### N-Oxide Impurity: 0.05%

Carboxylate impurity: Not detected

### Example 4

### Preparation of Crude Paliperidone Using N,N-Dimethyl Amino Pyridine (DMAP)

A 100 ml Flask equipped with a mechanical stirrer, reflux condenser was charged with CMHTP (7.15 gm), FBIP.HCl (5 gm), diisopropyl ethyl amine (6.5 gm), 4-N,N-dimethyl amino pyridine (0.125 gm) and methanol (50 ml) and stirred at room temperature. The reaction mixture was then refluxed at 60-70° C. for 8 to 10 hr. After completion of the reaction the reaction mixture was cooled to 0° C. and the product obtained was filtered and suck dried. The wet product was dried under vacuum at 50-55° C.

### Crude Paliperidone: 99.7%

### N-Oxide Impurity: 0.06%

Carboxylate impurity: Not detected

We claim:

1. An improved process for the preparation of Paliperidone which comprises the condensation of 3-(2-chloroethyl)-6,7,8,9-tetrahydro-9-hydroxy-2-methyl-4H-pyrrido[1,2-a]pyrimidi-4-one (CMHTP) (I) and 6-fluoro-3-piperidino-1,2-benisoxazol (FBIP) (II) or salt thereof in presence of tertiary organic amine and solvent.

2. The process as claimed in claim 1, wherein the solvent used is selected from C₁₋₆ straight or branched chain alcohols, ketone, ester, ether, polar aprotic solvent and mixture thereof.

3. The process as claimed in claim 2, wherein solvent used is methanol, ethanol, n-propyl alcohol, isopropyl alcohol, n-butyl alcohol, t-butyl alcohol, acetone, ethyl methyl ketone, ethyl acetate, methyl acetate, Dimethylformamide, dimethylacetamide, N-methylpyrrolidinone, diethyl ether, diisopropyl ether and methyl ether

4. The process as claimed in claim 3, wherein the alcohol used is methanol.

5. The process as claimed in claim 1, wherein the tertiary organic amine used is triethyl amine, diisopropyl ethyl amine, trimethyl amine, diethyl methyl amine, diisopropylmethyl amine and N-methyl morpholine.

6. The process as claimed in claim 5, wherein the tertiary amine base used is diisopropyl ethylamine.

7. A process as claimed in claim 1 further comprises, the purification of the product using alcohol, ketone or their mixture with water.

8. The process as claimed in claim 7, wherein ketone used is acetone, methyl ethyl ketone, methyl isobutyl ketone and alcohol used is isopropyl alcohol, methanol, ethanol and n-propyl alcohol.

9. The process as claimed in claim 8, wherein solvent used for purification is acetone and water mixture.

10. The process as claimed in claim 8, wherein solvent used for purification is isopropyl alcohol and water mixture.

11. A process of claim 1, further comprises the reaction is done in presence of phase transfer catalyst.

12. The process as claimed in claim 11, wherein the phase transfer catalyst used herein is trialkylphenylmethylammonium, tetraalkylammonium, tetraalkylphosphonium, tetraarylphosphonium halide, hydroxide and hydrogen sulfate.

13. The process as claimed in claim 12, wherein the phase transfer catalyst used herein is tetra n-butyl ammonium bromide.

14. A process of claim 1, further comprises the reaction is done in presence of N,N-dimethylaminopyridine.

15. The process as claimed in claims 7, 11 and 14, where in the final product of the present inventive substance has purity 99.6% or above.

16. The process as claimed in claims 7, 11 and 14, where in the final product of the present inventive substance has single maximum impurity less than 0.1%.
